(19) United States
(12) Patent Application Publication
(10) Pub. No.: US 2026/0083805 A1
(43) Pub. Date: Mar. 26, 2026

(54) COMPOSITION COMPRISING GLYCYRRHIZA URALENSIS EXTRACT AND METHOD AND USE THEREOF FOR SKELETAL MUSCLE GROWTH IN ANIMALS

(71) Applicant: ACADEMIA SINICA, Nankang, Taipei (TW)

(72) Inventors: Yu-Chuan LIANG, Taipei City (TW); Bor-Rung OU, Taichung City (TW)

(21) Appl. No.: 18/891,418

(22) Filed: Sep. 20, 2024

(51) Int. Cl. A61K 36/484; A61K 9/00; A61K 31/352; A61P 21/00; G01N 33/50

(52) U.S. Cl. CPC A61K 36/484; A61K 9/0053; A61K 31/352; A61P 21/00; G01N 33/5061

(57) ABSTRACT

The present disclosure provides a phytogenic-containing composition and applications thereof in agriculture. Specifically, the present disclosure relates to a composition comprising Glycyrrhiza uralensis extract, or an active compound isolated from the G. uralensis extract, a method for promoting skeletal muscle growth of animals by utilizing the composition, and use of the composition as a phytogenic feed additive for animals.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “113360_SEQ_LISTING.xml”, was created on Sep. 12, 2024, and is 7,000 bytes in size.

### FIELD OF INVENTION

The present disclosure relates to a phytogenic-containing composition and applications thereof in agriculture. Specifically, the present disclosure relates to a composition comprising Glycyrrhiza uralensis extract, or an active compound isolated from the G. uralensis extract, a method for promoting skeletal muscle growth of animals by utilizing the composition, and use of the composition as a phytogenic feed additive for animals.

### BACKGROUND OF THE INVENTION

Skeletal muscle is the largest tissue and represents approximately 40% of total body mass, and it is a multifunction tissue that plays a fundamental role in regular daily activities, including not only motor function and respiration, but also energy storage within the body in the form of proteins (Giordani, L. et al., Molecular Cell 2019, 74, 609-621; Tieland, M. et al., Journal of Cachexia, Sarcopenia and Muscle 2018, 9, 3-19). Furthermore, skeletal muscle represents the primary source of animal proteins in economically significant animals destined for human consumption. The growth and maturation of skeletal muscle has a direct impact on meat production (Matarneh, S. K. et al., Annual Review of Animal Biosciences 2021, 9, 355-377). Accordingly, skeletal muscle growth in livestock impacts meat quantity and quality.

Various feed additives, such as beta-agonists and anabolic steroids, are used in livestock to directly promote muscle growth (Niranjan, D. et al., Journal of Livestock Science (ISSN online 2277-6214) 2023, 14, 53-64). Beta-agonists increase skeletal muscle mass and decrease fat mass in animals by stimulating muscle protein synthesis (Elliott, C. T. et al., Vet Res Commun 1993, 17, 459-46). In addition, recombinant bovine somatotropin enhances growth rates, feed conversion, and lean meat in beef cattle (Moseley, W. M. et al., J Anim Sci 1992, 70, 412-425). However, these feed additives have been known to have disadvantages and food safety problems. Beta-agonist residues in animal meats and organs raise food safety concerns (Martinez-Navarro, J. F., The Lancet 1990, 336, 1311), and some beta-agonists also generate risks of tachycardia, hypertension, and cardiac hypertrophy potentially leading to heart failure in animals and humans (Lehmann, S. et al., Forensic Science International 2019, 303, 109925). On the other hand, anabolic steroids cause endocrine disruptions, and are reported to be associated with carcinogenic, immunotoxic, mutagenic, and teratogenic effects, leading to irreversible consequences (Skoupi, K. et al., Animals 2022, 12, 2115; Hirpessa, B. B. et al., Journal of Food Quality 2020, 2020, 1-12). It is of utmost importance to search for feed additives that can promote muscle growth of animals or livestock without adverse effects. Hence, there remains a need in the field for feed additives that not only are safe but also directly and significantly promote muscle growth of animals or livestock.

Phytogenic additives are derived from various plant parts, such as leaves, roots, seeds, flowers, buds, or bark, and their extracts. Some phytochemical compounds have a long history of use in human medicine and are known for their pharmacological effects (Martel, J. et al., Trends in Biochemical Sciences 2020, 45, 462-471; Dhama, K. et al., Curr Drug Metab 2018, 19, 236-263; Abdelli, N. et al., Animals 2021, 11, 3471). Owing to their natural and safe properties, phytogenic feed additives become more popular as natural alternatives than conventional additives, such as antibiotics and growth-promoting hormones (Abdelli, N. et al., Animals 2021, 11, 3471; Upadhaya, S. D. et al., Annals of Animal Science 2017, 17, 929-948; Windisch, W. et al., Journal of Animal Science 2008, 86, E140-E148). The majority of phytogenic feed additives are recognized for their diverse biological activities in antimicrobial, antioxidant, anti-inflammatory, gut health and digestive-enhancing properties to promote growth performance indirectly (Mnisi, C. M. et al., Applied Sciences 2023, 13, 99; Basiouni, S. et al., Veterinary Sciences 2023, 10, 55; Marks, H. et al., Microorganisms 2022, 10, 629). However, only a limited number of phytogenic feed additives are addressed to promote skeletal muscle growth.

Myostatin (MSTN) is a secreted protein that serves as an inhibitory factor in the development of skeletal muscle. MSTN is a member of the transforming growth factor (TGF) family and has important functions in skeletal muscle, and therefore, it has become an important therapeutic target due to its crucial involvement in several disorders. Furthermore, the MSTN gene is remarkably conserved across mammals, and mutations resulting in its loss-of-function have been observed in various species, including cattle, sheep, pigs, rabbits, and humans. These mutations result in increased skeletal muscle mass and the emergence of a “double-muscle” phenotype[21-23]. Therefore, inhibition of MSTN gene expression should potentially enhance skeletal muscle development in both muscle cells and animals.

To enhance skeletal muscle growth of livestock, phytogenic feed additive may be a potential alternative solution to inhibit MSTN activity in animals. There is also a need to establish a systematic screening platform or method to evaluate the activity of phytogenics through MSTN activity and thereby identify potential phytogenics with MSTN-inhibiting activity to improve the growth performance of animals or livestock.

### SUMMARY OF THE INVENTION

In one aspect, the present disclosure provides a composition comprising Glycyrrhiza uralensis extract or an active compound isolated from the G. uralensis extract, for use in promoting skeletal muscle growth in an animal in need thereof. In particular, the composition of the present disclosure can be used for enhancing growth performance of an animal.

In another aspect, the present disclosure provides a method for promoting skeletal muscle growth in an animal in need thereof, comprising administering to the animal a composition comprising G. uralensis extract or an active compound isolated from the G. uralensis extract.

In another aspect, the present disclosure provides use of a composition comprising G. uralensis extract or an active compound isolated from the G. uralensis extract, as a feed additive for an animal in need thereof. In particular, the G. uralensis extract or an active compound isolated from the G. uralensis extract is administered in an effective amount to promote skeletal muscle growth in the animal in need thereof.

In another aspect, the present disclosure provides a feed additive for animals, wherein the feed additive comprises a composition comprising G. uralensis extract or an active compound isolated from the G. uralensis extract. In some embodiments, the feed additive is a phytogenic feed additive and can provide various benefits to the animal, including promoting skeletal muscle growth and enhance growth performance without causing any adverse effect or toxicity in animals.

In one embodiment, the G. uralensis extract in the composition of the present disclosure is present in an amount effective to promote the skeletal muscle growth in an animal in need thereof. In further embodiments, the G. uralensis extract may be administered to the animal at a concentration ranging from about 3 μg/mL to about 50 μg/mL, preferably from about 5 μg/mL to about 30 μg/mL, more preferably from about 6.25 μg/mL to about 25 μg/mL, even more preferably 6.25 μg/mL to about 12.5 μg/mL, and most preferably from about 12.5 μg/mL to about 25 μg/mL. In a preferred embodiment, the G. uralensis extract is administered at a concentration of about 6.25 μg/mL, about 12.5 μg/mL, or about 25 μg/mL, more preferably about 12.5 μg/mL or about 25 μg/mL, and most preferably about 25 μg/mL.

In another embodiment, the composition comprising G. uralensis extract is prepared and formulated into a feed additive for use in an animal feed. In particular, the animal feed comprises the composition comprising Glycyrrhiza uralensis extract in an amount ranging from about 0.01% to about 0.5%, more preferably from about 0.01% to about 0.1%, even more preferably from about 0.01% to about 0.05%, and most preferably from about 0.05 to about 0.1%, by weight based on the total weight of the animal feed. In a preferred embodiment, the animal feed comprises the composition comprising G. uralensis extract in an amount of about 0.01%, about 0.05% or about 0.1% by weight of the animal feed.

In the method or use of the present disclosure, the amount of G. uralensis extract in the composition of the present disclosure may be administered in an amount effective to promote the skeletal muscle growth in an animal in need thereof. In further embodiments, the effective amount of G. uralensis extract may be in a range of from about 3 μg/mL to about 50 μg/mL, preferably from about 5 μg/mL to about 30 μg/mL, more preferably from about 6.25 μg/mL to about 25 μg/mL, even more preferably 6.25 μg/mL to about 12.5 μg/mL, and most preferably from about 12.5 μg/mL to about 25 μg/mL. In a preferred embodiment, the G. uralensis extract is administered at a concentration of about 6.25 μg/mL, about 12.5 μg/mL, or about 25 μg/mL, more preferably about 12.5 μg/mL or about 25 μg/mL, and most preferably about 25 μg/mL.

In the method or use of the present disclosure, the amount of G. uralensis extract as a feed additive in an animal feed may be in a range of from about 0.01% to about 0.5%, more preferably from about 0.01% to about 0.1%, even more preferably from about 0.01% to about 0.05%, and most preferably from about 0.05% to about 0.1%, by weight based on the total weight of the animal feed. In a preferred embodiment, the animal feed comprises the composition comprising G. uralensis extract in an amount of about 0.01%, about 0.05% or about 0.1% by weight of the animal feed.

In a further embodiment, the G. uralensis extract is obtained from dried G. uralensis roots or the dried plants without roots. In a further embodiment, the G. uralensis extract is prepared by an extraction method comprising extraction with an aqueous ethanol solution. In a more preferred embodiment, the G. uralensis extract is extracted with aqueous ethanol solution, preferably 95% ethanol solution. In a most preferred embodiment, the G. uralensis extract is prepared by mixing the G. uralensis plant material grounded into powders (e.g., with a size of 20 Mesh) with 10 parts of 95% ethanol solution with stirring for 24 h, filtering the liquid extract through a sieve (e.g., with a size of 200 Mesh) and then concentrating the resultant by evaporation to obtain the plant extract.

In another aspect, the present disclosure provides a screening method for a potential phytogenic in promoting skeletal muscle growth in animals, wherein the activity of the phytogenic is evaluated through MSTN activity and the potential myogenesis-related phytogenic is identified. In one embodiment, the screening method is carried out in a systematic rodent myoblast screening platform by targeting myostatin (MSTN) promoter activity to identify a phytogenic involving myogenesis enhancement. In a further embodiment, the systematic screening platform is used to monitor myostatin promoter activity in rat L8 myoblasts. In a further embodiment, the phytogenic exhibiting an inhibitory effect on MSTN expression is determined to have the potency of promoting skeletal muscle growth of the animals.

In one embodiment, the screening method for a potential phytogenic in promoting skeletal muscle growth in animals comprises:
(a) preparing a plant extract from a plant or plant part of interest;
(b) preparing the animal cells by transfecting the cells with a plasmid containing a myostatin-luciferase (MSTN-Luc) reporter gene;
(c) treating the transfected animal cells from (b) with the herbal extract from (a) and maintaining the treated cells in a culture medium under a condition sufficient for cell proliferation;
(d) measuring cell proliferation of the treated cells from (c);
(e) measuring the luciferase activity of the treated cells from (c);
(f) comparing the cell proliferation measured in (d) with a vehicle control under the same condition;
(g) comparing the luciferase activity measured in (e) with a control under the same condition; and
(h) determining the plant extract tested as effective phytogenic for promoting skeletal muscle growth by the increased cell proliferation and higher inhibition of luciferase activity exhibited by the cells treated with the plant extract.

In the screening method, the animal cells prepared in (b) are rat L8 myoblast cell line. The cell proliferation of the treated cells in (d) is measured by performing MTT (3-(4,5-dimethylthiazolyl-2)-2,5-diphenyltetrazolium bromide) colorimetric assay, in which MTT reagent is added to the treated cells cultivated in a culture medium, preferably supplemented DMEM, at 37° C. for 4 h, followed by adding dimethyl sulfoxide (DMSO) to detect optical density at 595 nm. Furthermore, the luciferase activity of the treated cells incubated in a culture medium, preferably a supplemented DMEM, is measured in (e) after 24 h treatment. The decrease in the MSTN mRNA level, luciferase activity and ratio of luciferase activity to cell proliferation in the treated cells are used to determine the inhibitory effect of the plant extract.

The present invention provides a surprising discovery that the composition comprising Glycyrrhiza uralensis extract, or an active compound isolated from the G. uralensis extract, can promote skeletal muscle growth of animals particularly through inhibition of MSTN expression and thus significantly enhanced the growth performance of animals after being administered as a feed additive to the animals. Specifically, the enhanced growth performance of animals includes the increased body weight, carcass weight, and lean content of the animals. Furthermore, the composition of gut microbiota in the animals after being administered with the feed additive comprising the G. uralensis extract was not influenced.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purposes of illustrating the invention, shown in the drawings are embodiments which are presently preferred.

In the drawings:

FIG. 1A is a HPLC chromatogram showing peaks of cudraflavone-C purified and identified from fraction (G9) in G. uralensis extract at UV 254 nm, 280 nm and 300 nm. FIG. 1B is a LC-MS/MS chromatogram showing peaks of cudraflavone-C purified and identified from fraction (G9) in G. uralensis extract at UV 254 nm.

FIG. 2A is a plasmid map of a myostatin promoter-reporter plasmid, pMetLuc2-MSTN, constructed by inserting the 5′-flanking region (2.46 kb) upstream of the translation start site of the MSTN gene into pMetLuc2 plasmid. FIG. 2B is an electrophoresis gel showing bands in different lanes (Lane 1: pMetLuc2 plasmid after BglII digestion; Lane 2: pMetLuc2-MSTN plasmid after BglII digestion; Lane 3: plasmid after BglII and BamH1 digestions; Lane 4: PCR amplification product of the 5′-flanking region (2.46 kb) upstream of the translation start site of MSTN gene; Lane M: 1-kb DNA ladder).

FIG. 3A-FIG. 3K show the luciferase activity assay on L8 MSTN-Luc cells treated with dexamethasone (FIG. 3A) or various herbal extracts including Andrographis paniculate (FIG. 3B), Centella asiatica (FIG. 3C), Glycyrrhiza uralensis (FIG. 3D), Gynostemma pentaphyllum (FIG. 3E), Platycodon grandifloras (FIG. 3F), Polygonum chinense (FIG. 3G), Portulaca oleracea (FIG. 3H), Saururus chinensis (FIG. 3I), Smilax china (FIG. 3J) and Taraxacum campylodes (FIG. 3K). L8 MSTN-Luc cells luciferase activity was measured after 24 h treatment, and each data point represents the mean±SEM. Different letters (a-d) denote significant differences (p<0.05) amount treatments.

FIG. 4A-FIG. 4J show the effect of various herbal extracts on L8 MSTN-Luc cell proliferation. L8 MSTN-Luc cells were treated with various herbal extracts, including Andrographis paniculate (FIG. 4A), Centella asiatica (FIG. 4B), Glycyrrhiza uralensis (FIG. 4C), Gynostemma pentaphyllum (FIG. 4D), Platycodon grandifloras (FIG. 4E), Polygonum chinense (FIG. 4F), Portulaca oleracea (FIG. 4G), Saururus chinensis (FIG. 4H), Smilax china (FIG. 4I) and Taraxacum campylodes (FIG. 4J) in different concentrations or with 1% ethanol as vehicle control. L8 MSTN-Luc cell proliferation was measured using MTT assay. Different letters (a-d) denote significant differences (p<0.05) among treatments. Each data point represents the mean±SEM from three independent experiments.

FIG. 5A-FIG. 5D show the effect of G. uralensis extract (GUE) on MSTN mRNA level and MSTN promoter activity in L8 MSTN-Luc cells. The effect of GUE on MSTN mRNA level was determined by reverse transcription-polymerase chain reaction (RT-PCR)(FIG. 5A). The relative ratio of MSTN mRNA level to internal control was calculated (FIG. 5B). The effect of GUE on cell proliferation was investigated by MTT assay. MSTN promoter activity was measured by luciferase activity assay (FIG. 5C). The ratio of luciferase activity to cell proliferation (FIG. 5D) was used to evaluate GUE inhibitory effect. Different letters (a-d) denote significant differences (p<0.05) among treatments. Each data point represents the mean±SEM from three independent experiments.

FIG. 6A-FIG. 6B show the systematic analysis of major active fractions of G. uralensis extract. The ethanolic extract of G. uralensis was purified by Process-Scale Liquid Column Chromatography and separated into ten fractions (FIG. 6A). The bioactive fraction was identified by MSTN gene reporter assay in L8 MSTN-Luc cells treated with various GUE concentrations (FIG. 6B). The results are indicated as the ratio of luciferase activity to cell proliferation.

FIG. 7A-FIG. 7C show the chemical profile of the MSTN-inhibitory fraction (G9) in G. uralensis extract. The chemical profile of G. uralensis active fraction (G9) was analyzed using the RP-18 column and detected with DAD detection at 210-540 nm (FIG. 7A). Peaks of bioactive compounds and resulting chemical structures were identified as shown (FIG. 7B). Dose-response curves of three individual phytocompounds and the G9 fraction for MSTN suppression activity (FIG. 7C). Each data point represents the mean±SEM.

FIG. 8A-FIG. 8C show the effect of GUE as a feed additive on body weight in growth-finishing pigs. Flow chart shows the timeline for evaluating of GUE effect as a feed additive on growth-finishing pigs (FIG. 8A). After weaning, pigs were randomized into four groups, including standard control diet (CTL), low GUE concentration (LGUE), medium GUE concentration (MGUE) and high GUE concentration (HGUE) treatment of 10 animals each group. GUE as a feed additive was fed for 18 weeks. Body weight (FIG. 8B) and body gain weight (FIG. 8C) in each treatment group was monitored before and after 18-week treatment. Asterisks indicate the significant difference (p<0.05) in body gain weight between HGUE and CTL groups in the same treatment week. Each data point represents the mean±SEM.

FIG. 9A-FIG. 9G show the effect of GUE as a feed additive on carcass characteristics of pigs. Four groups of weaned pigs were fed standard control diet (CTL), or standard diet containing low GUE concentration (LGUE), medium GUE concentration (MGUE) and high GUE concentration (HGUE) for 18 weeks. Carcass weight (FIG. 9A), carcass percentage (FIG. 9B), carcass length (FIG. 9C), as well as the percentage of lean (FIG. 9D), fat (FIG. 9E), bone (FIG. 9F) and skin (FIG. 9G) of six representative pigs from each groups, were measured and calculated at the end of 18 weeks. Each data point represents the mean±SEM. Different letters denote statistical significance (p<0.05) among groups.

FIG. 10 shows the effect of GUE as a feed additive on gut microbiota in pigs. The bacterial composition at the genus level in the guts of pigs fed control diet (CTL), or diet containing low GUE concentration (LGUE), medium GUE concentration (MGUE) and high GUE concentration (HGUE) for 18 weeks was shown from three representative pigs in each groups were pyrosequenced and the data were analyzed at the end of 18 weeks.

### DETAILED DESCRIPTION

Phytogenics are heterogeneous compounds with different biological activities considered to have some similar benefits as antibiotic growth promoters. Numerous herbal products have been demonstrated to have beneficial effects and medicinal properties, including antimicrobial, antioxidant, anti-inflammatory, and immunomodulatory activities, without adversely affecting growth and feed efficiency, and are therefore used as growth-promoting feed additives in livestock production. There is an increasing preference for natural products due to being considered to have less undesirable side effects than synthetic products. The development of plant-based feed additives has become significant for the healthy development of animal husbandry and the improvement of animal product quality.

Plant-based or phytogenic feed additives have been shown to exhibit antioxidant and antimicrobial properties, and also support gut microbiota and functions to improve animal growth performance. However, little is known about whether this action was modulated through myogenesis directly during animal growth. In the present disclosure, myogenesis-related phytogenics were screened through a systematic MSTN expression screening platform using L8 cells. MSTN belongs to the TGF-β superfamily and is a secreted protein that acts as a negative regulator in the process of skeletal muscle development. MSTN exhibits a high degree of conservation across mammals including cattle, sheep, pigs, rabbits, and humans. Loss-of-function mutations are associated with increased skeletal muscle mass. Hence, myostatin plays a pivotal role in regulating myogenesis. Inhibition of MSTN expression may have the potential to improve skeletal muscle growth in animals. Presently, a MSTN inhibitor, the monoclonal antibody known as bimagrumab binds to and restrains the MSTN activity to result in increased muscle mass and strength. Clinical trials demonstrated that bimagrumab effectively enhanced muscle mass and function in humans suffering from muscle-wasting conditions, such as sarcopenia and inclusion body myositis. Additionally, MSTN inhibitors could generate potential side effects, including the increased risk of tumor development, thus further research is necessary to evaluate the safety issue.

In livestock industry, there is a growing demand for feed additives that are safe, convenient, and efficient to improve animal growth and performance. Therefore, the plant-based or phytogenic feed additive is a better strategy than the monoclonal antibody administration to improve animal growth. To rationalize the use of phytogenics, this established systematic MSTN expression screening platform was used to screen various herbal plant extracts which were not only related to skeletal muscle diseases or its regulation, but also were traditionally used as food ingredients and botanical medicine for humans and animals.

Plant-derived products have traditionally been used as a source of medicine. For example, Glycyrrhiza is a genus of about 18 accepted species in the legume family (Fabaceae), with a sub-cosmopolitan distribution in Asia, Australia, Europe, and the Americas. G. uralensis, also known as Chinese liquorice, is a flowering plant native to Asia, which is used in traditional Chinese medicine. Liquorice root is one of the 50 fundamental herbs used in traditional Chinese medicine.

In Chinese medicine, anti-inflammatory liquorice root has been used for centuries for many uses such as for treating coughs and colds, gastrointestinal issues, and female reproductive issues. Liquorice root has been used in tandem with other herbs and remedies to enhance their effects and essentially guide the other herbs to where they would be most beneficial. Liquorice root is a complex mixture of compounds, and researchers have isolated 134 different compounds in the glabra variety and 170 different compounds in G. uralensis. There are at least four main types of compounds found in licorice root: flavonoids, coumarins, triterpenoids and stilbenoids. However, none of the compounds or extracts derived from Liquorice has been studied and identified for use in promotion of skeletal muscle growth in animals.

The present invention provides a composition comprising G. uralensis extract or an active compound isolated from G. uralensis extract identified through a systematic rodent myoblast screen platform, which can be used as a feed additive in an animal feed which exhibits beneficial effect on growth performance of the animal.

In accordance with the embodiment of the present invention, the composition comprising G. uralensis extract as an active ingredient is provided. In some embodiments, the active compounds isolated from G. uralensis extract are the active fractions of G. uralensis extract identified by means of extraction with ethanol. According to the chemical structures, the identified active fractions of Glycyrrhiza uralensis extract are gancaonin G, cudraflavone-C and licoisoflavone A, which exhibit an inhibitory effect on MSTN expression and are determined as effective in promoting skeletal muscle growth in animals.

The following description when being read with the accompanying drawings are made to clearly exhibit the above-mentioned and other technical contents, features and effects of the present disclosure. As the contents disclosed herein should be readily understood and can be implemented by a person skilled in the art, all equivalent changes or modifications which do not depart from the concept of the present disclosure should be encompassed by the appended claims.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

As used in the description of the invention and the appended claims, the singular forms “a”, “an” and “the” are intended to include the plural forms as well, unless the context clearly indicates otherwise. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, the term “about” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result, or embodiment, the term “about” means within one standard deviation per the practice in the art, or a range of up to 1%, 2%, 3%, 4%, 5%, 6%, 7%, 8%, 9%, 10%, 11%, 12%, 13%, 14%, 15%, 16%, 17%, 18%, 19%, or 20%.

As used herein, the terms “comprises,” “comprising,” “includes,” “including,” “has”, “having”, “contains”, “containing”, “characterized by”, or any other variation thereof, are intended to cover a non-exclusive inclusion, subject to any limitation explicitly indicated. For example, a composition, mixture, process, or method that comprises a list of elements is not necessarily limited to only those elements but may include other elements not expressly listed or inherent to such composition, mixture, process, or method.

The transitional phrase “consisting of” excludes any elements, steps, or ingredients not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

Where applicants have defined an invention or a portion thereof with an open-ended term such as “comprising”, it should be readily understood that (unless otherwise stated) the description should be interpreted to also describe such an invention using the term “consisting of”.

The use of the term “or” in the claims is used to mean “and/or” unless explicitly indicated to refer to alternatives only or the alternatives are mutually exclusive, although the disclosure supports a definition that refers to only alternatives and “and/or.”

The term “component” with regard to a composition is herein understood as a part of a composition, which may include one or more further components and excipients. The composition of the present disclosure comprises at least Glycyrrhiza uralensis extract or an active compound isolated from the Glycyrrhiza uralensis extract, but may further include biological components, primarily phytogenic components and excipients.

In the present disclosure, the term “animal” as used herein refers to not only domesticated animals such as husbandry animals, pets, and humans, more particularly pigs, and other infant husbandry animals. In the present disclosure, pigs are exemplary animals and have a significant economic value in animal husbandry. However, the particular aspects of the present disclosure may also include other husbandry animals (such as cattle, goats, sheep, and horses), pets and humans.

In the present disclosure, the term “feed” used herein is used for “feed and food” collectively or feed only. If solely human consumption is meant, the term “food” is used.

The term “gut microbiota”, as used herein according to the context, refers to the microorganisms, including bacteria, archaea, fungi, and viruses, that live in the digestive tracts of animals.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

The term “liquid chromatography” used herein refers to a process in which a biological/chemical mixture carried by a liquid can be separated into components as a result of differential distribution of the components as they flow through (or into) a stationary liquid or solid phase. Non-limiting examples of liquid chromatography include reversed-phase liquid chromatography, ion-exchange chromatography, size exclusion chromatography, affinity chromatography, hydrophobic interaction chromatography, hydrophilic interaction chromatography, or mixed-mode chromatography. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

### EXAMPLES

The Examples are designed to further illustrate the present invention and serve a better understanding. They are not to be construed as limiting the scope of the invention in any way.

### Materials and Methods Used in the Examples

Unless stated otherwise, the following materials and methods were used in the examples.

### Materials

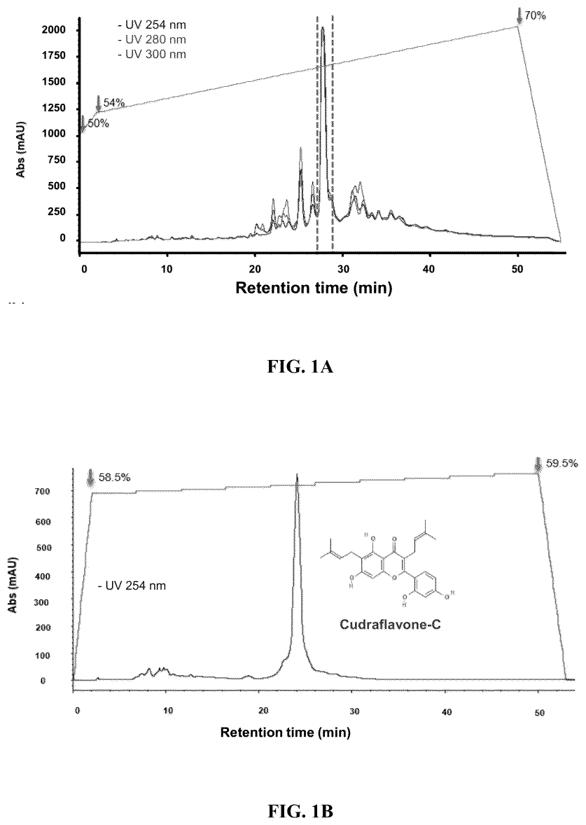

Dried Glycyrrhiza uralensis and Platycodon grandiflorus roots, as well as dried plants without roots, from Andrographis paniculata, Centella asiatica, Gynostemma pentaphyllum, Polygonum chinense, Portulaca oleracea, Saururus chinensis, Smilax china, and Taraxacum campylodes were procured from a reputable Chinese medicinal herb store in Taiwan (July 2020). Taq DNA polymerase was obtained from Kapa Biosystems (Roche, Basel, Switzerland). pMetLuc2 plasmid DNA and Ready-To-Glow™ Secreted Luciferase Reporter System were obtained from Clontech (Mountain View, CA, USA). Bovine serum albumin (BSA), T4 DNA ligase, and restriction enzymes (BglII and BamHI) were purchased from Promega (Madison, WI, USA). Gancaonin G and licoisoflavone A were obtained from ChemFaces (Wuhan, Hubei, China). Cudraflavone-C was purified in Dr. Liang's laboratory (FIG. 1A and FIG. 1B). Methylthiazoletetrazolium (MTT), dimethyl sulfoxide (DMSO) and Geneticin™ selective antibiotic (G418 sulfate) were obtained from Sigma (St Louis, MO, USA). Dulbecco's Modified Eagle Medium (DMEM) and fetal bovine serum (FBS) were obtained from Hyclone (Logan, UT, USA). Ethanol was obtained from J. T. Baker (Phillipsburg, NJ, USA). In this study, all other chemicals and solvents utilized were of reagent or high-performance liquid chromatography (HPLC) quality.

### Plant Extract Preparation and Cell Transfection

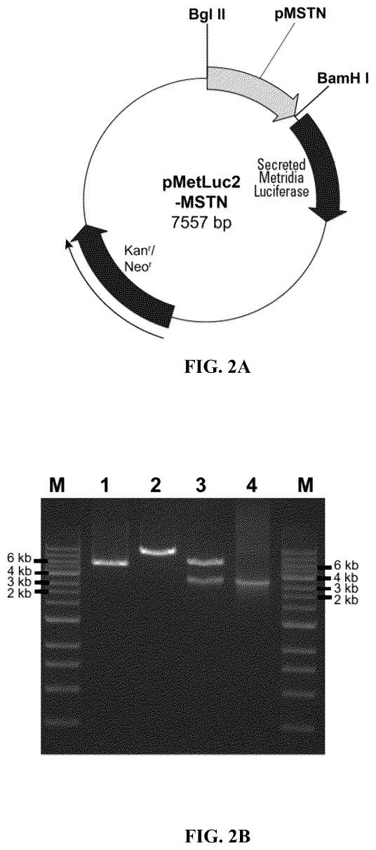

The dried herbal plant material was ground into powder (20 Mesh), then mixed with 10 parts of 95% ethanol and stirred for 24 h. The liquid extract was filtered through a sieve (200 Mesh) and subsequently concentrated using a rotary vacuum evaporator. The crude herbal extracts were used for in vitro tests or fractionated to different fractions later. The Rat L8 myoblast cell line was obtained from American Type Culture Collection (Manassas, VA, USA), maintained in DMEM supplemented with 10% FBS, penicillin (100 U/mL), streptomycin (100 μg/mL), sodium pyruvate (1 mM), and glutamate (292 μg/mL) and incubated at 37° C. under 5% CO₂. The 50-flanking region (2.46-kb) upstream of the translation start site of the MSTN gene (GenBank accession no. AY204900) was amplified from mouse genomic DNA (Salerno, M. S. et al., American journal of physiology-Cell physiology 2004, 287, C1031-C1040) by polymerase chain reaction (PCR) with a pair of the specific primers containing BglII and BamHI sites, i.e., MSTN-FF: 5′-ATAAGATCTCCTTTTTAAGTCCTAAGTCACACGG-3′ (SEQ ID NO: 1) and MSTN-FR: 5′-ATAGGATCCCCAGGGAGTCCTGTATACTG-3′ (SEQ ID NO: 2). The pMetLuc2-MSTN (FIG. 2A) reporter plasmid was constructed by inserting this 2.46-kb MSTN promoter into pMetLuc2 plasmid. The pMetLuc2-MSTN L8 cells (“L8 MSTN-Luc cells”) used in this study were derived by stable transfection of pMetLuc2-MSTN and selected with G418.

### MTT Assay

For MTT assay, stable transfected L8 MSTN-Luc cells were counted with a hemocytometer (Hausser Scientific, PA, USA), and 2×10⁴ cells of were seeded onto each well on a 96-well plate in DMEM containing 5% FBS. The experiment was performed when the cells reached 80% confluence. Various concentrations of herbal extracts were used to treat the cells for 24 h at 37° C., and then the cell proliferation was determined by MTT colorimetric assay (Yeh, J. Y.; et al., Cell Mol Life Sci 2002, 59, 1972-1982). Briefly, MTT reagent (2 mg/mL) was added to each well and incubated for 4 h at 37° C. After adding dimethyl sulfoxide (DMSO) to each well, the OD₅₉₅ of each well was measured by BioTek Powerwave XS microplate reader (Winooski, VT, USA).

### Luciferase Assays

For luciferase activity, 2×10⁴ cells of stable L8 MSTN-Luc cells were seeded onto 96-well plates with DMEM containing 5% FBS. Secreted Metridia luciferase activity was measured using the Ready-To-Glow™ Secreted Luciferase Reporter Systems after 24-h treatment of various herbal extracts. The culture medium was removed to mix with luciferase substrate and VICTOR3 multilabel counter (PerkinElmer, Turku, Finland) was used to measure luciferase activity. The luciferase activity was normalized with cell proliferation prior to statistical analysis.

### Reverse Transcription-Polymerase Chain Reaction (RT-PCR)

Total RNA was extracted from L8 cells using the Trizol Reagent (Invitrogen, Carlsbad, CA, USA) according to the manufacturer's instructions, and then reverse transcribed into complementary DNA using Revert Aid Reverse Transcriptase (Thermo Fisher Scientific, Waltham, MA, USA) and oligo(dT) as primer. The PCR was performed by Taq DNA polymerase using the primers specific to MSTN, i.e., MSTN-F: 5′-CCAGGCACTGGTATTTGGCA-3′ (SEQ ID NO: 3) and MSTN-R: 5′-AAGTCTCTCCGGGACCTCTT-3′ (SEQ ID NO: 4), and using the primers specific to β-actin, i.e., Bactin-F: 5′-GGTTGGTATGGGCCAGAAAGA-3′ (SEQ ID NO: 5) and Bactin-R: 5′-TGGTGACAATACCGTGTTCAATG-3′ (SEQ ID NO: 6). Amplification was conducted using a GeneAmp PCR System 9700 (Applied Biosystems, Foster City, CA, USA) under the following conditions: an initial cycle at 94° C. for 5 min, followed by 35 cycles at 94° C. for 30 s, 55° C. for 30 s, and 72° C. for 30 s. The cycling was ended with a final elongation step at 72° C. for 5 min. The 3-actin was used as the internal control. Amplicons after 2% agarose gel electrophoresis were visualized by a Doc Print System (Vilber Lourmat, Marne-la-Vallée, France), and the relative band intensities were assessed using the ImageJ software (NIH, Bethesda, MD, USA).

Glycyrrhiza uralensis Extract (“GUE”) Preparation

The grinded G. uralensis (1 kg) was extracted using 95% ethanol solution for 24 h prior to evaporation. After evaporation, the extract was applied to a process-scale liquid column chromatography system (Isolera Flash Puri, Biotage, Uppsala, Sweden). The separation was performed using a C18 column (KP-C18-HS column, Biotage) and the solvent gradient was 95% EtOH in H₂O, and the extract was separated into 10 fractions under UV 245 nm. The major components of G9 were purified and isolated by using semi-preparative HPLC (Agilent, Palo Alto, CA, USA) on a RP-18 column (250×10 mm, Phenomenex, Torrance, CA, USA) at a flow rate of 1.0 mL/min, detected at UV 254 nm, 280 nm, and 300 nm. The chemical structures of the three compounds were determined through spectroscopic analysis. Electrospray ionization mass spectrometry data were acquired using an LCQ Advantage mass spectrometer (Thermo Finnigan, San Jose, CA, USA), while nuclear magnetic resonance spectra were recorded using Avance 500 and 300 MHz FT nuclear magnetic resonance spectrometers (Bruker, Bremen, Germany) at 500 MHz (¹H) and 75 MHz (¹³C). These three major compounds of G9 were identified as gancaonin G, cudraflavone C, and licoisoflavone A by comparing their NMR, MS data, and optical rotations with those reported previously (Tahara, S. et al., Phytochemistry 1989, 28, 901-911; He, J. et al., J. Nat. Prod. 2006, 69, 121-124; Dej-adisai, S. et al., Arch. Pharm. Res. 2014, 37, 473-483).

### Animal Study

To investigate the beneficial effect of the GUE as a feed additive, the growth-finishing pigs were fed standard diet or the diet containing GUE in the experimental ranch at Tunghai University (Taichung, Taiwan). Twenty barrows and twenty gilts ([Landrace×Yorkshire]×Duroc) with an initial average body weight (BW) of 12.24±1.42 kg were randomly distributed among four treatment groups (control, and low (0.01%), medium (0.05%) and high (0.1%) concentrations of GUE with 10 pigs each group). All animals were monitored according to the previous standard procedure (Tung, H. Y. et al., Lab. Anim. 2018, 52, 186-195) and were acclimatized with standard diet (Fwusow Industry Co. ltd., Taichung, Taiwan) and free access to water for one week prior to the experiment. Mortality, body weight, feed and water intake were recorded weekly. At the end of experiment, hot carcass weights were recorded and used to calculate the carcass percentage. The length of each carcass spanning from the posterior edge of the symphysis pubis to the anterior edge of the first rib was measured, and the skin, bone, legs, shoulders, loins, bellies, tenderloins, backfat and neck fat were collected using the simplified EC-reference method (Branscheid, W. et al., Fleischwirtschaft 1990, 70, 565-567). All experimental protocols received approval from the Institutional Animal Care and Use Committees in Tunghai University (Protocol no. 107-38).

### Pyrosequencing and Data Analysis

The gut bacterial DNA extracted from pig feces in each treatment group at the end of experiment were used as templates for PCR amplification with 16S rRNA primers. The 16S rRNA amplicons for the illumine MiSeq platform (Illumina, San Diego, California, USA). In order to prepare library, the primer pair sequences target the V3 and V4 region, which generate a single amplicon of approximately 460 base pairs. A limited cycle PCR are performed to amplify the V3 and V4 region, and Illumina sequencing adapters are incorporated along with dual-index barcodes into the target amplicon. With the complete set of Nextera XT indices, it is possible to combine and sequence up to 96 libraries concurrently. Employing the MiSeq platform with paired 300-base pair reads and MiSeq v3 reagents, the ends of each read are overlapped to generate high-quality, full-length reads of the V3 and V4 region in a single 65-h run. The results of 16S rRNA gene amplicons sequencing will be analyzed by MiSeq Reporter (Illumina), BaseSpace (Illumina) and Greengenes database (Lawrence Berkeley National Laboratory, Berkeley, CA, USA) to complete taxonomy and classification of microbiota GUE-treated or non-treated in different stages of pigs.

### Statistics

The data are presented as mean±Standard Error of the Mean (SEM) and examined for equal variance and normal distribution prior to statistical analysis using analysis of variance (ANOVA). A significance level of 0.05 was adopted. Fisher's least significant difference (LSD) test was used for group comparisons.

### Example 1: Phytogenic Screening by Using Systematic Rodent Myoblast Screen Platform

In this study, myostatin-luciferase (MSTN-Luc) reporter gene was successfully transfected into L8 cells and the stable clone (L8 MSTN-Luc cells). The L8 MSTN-Luc cells were treated with dexamethasone at different concentrations (i.e., 0, 25, 50 and 100 M) or various herbal extracts at different concentrations (i.e., 0, 6.25, 12.5 and 25 μg/mL), and 1% ethanol was used as vehicle control. The herbal extracts selected to be screened in this study are the common herbal plants related to skeletal muscle diseases or regulation, including Andrographis paniculate, Centella asiatica, Glycyrrhiza uralensis (“GUE”), Gynostemma pentaphyllum, Platycodon grandifloras, Polygonum chinense, Portulaca oleracea, Saururus chinensis, Smilax china and Taraxacum campylodes. In the luciferase activity assay, the luciferase activities of the L8 MSTN-Luc cells treated with dexamethasone or each of the herbal extracts were measured after 24 h treatment to determine the effect of various herbal extracts on L8-MSTN-Luc cell proliferation.

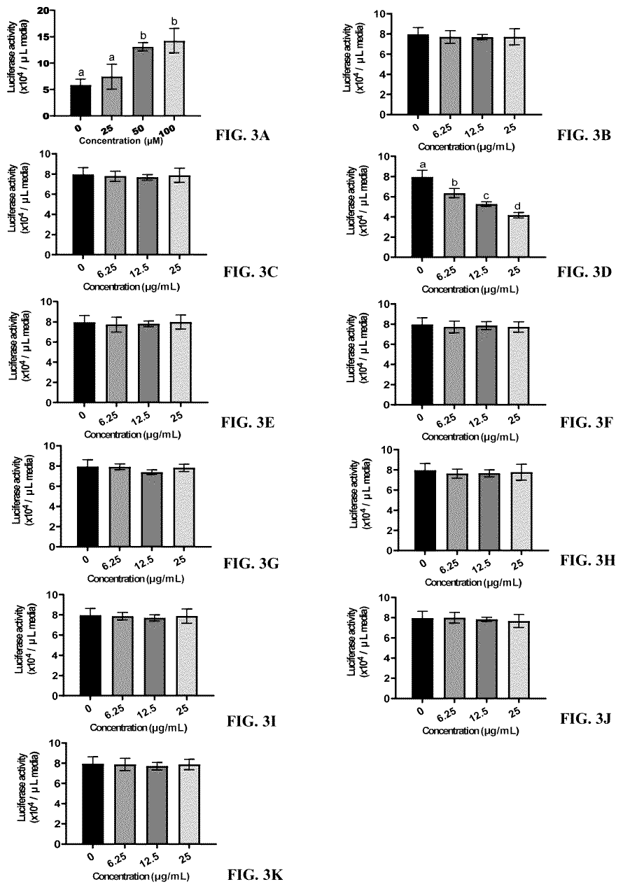

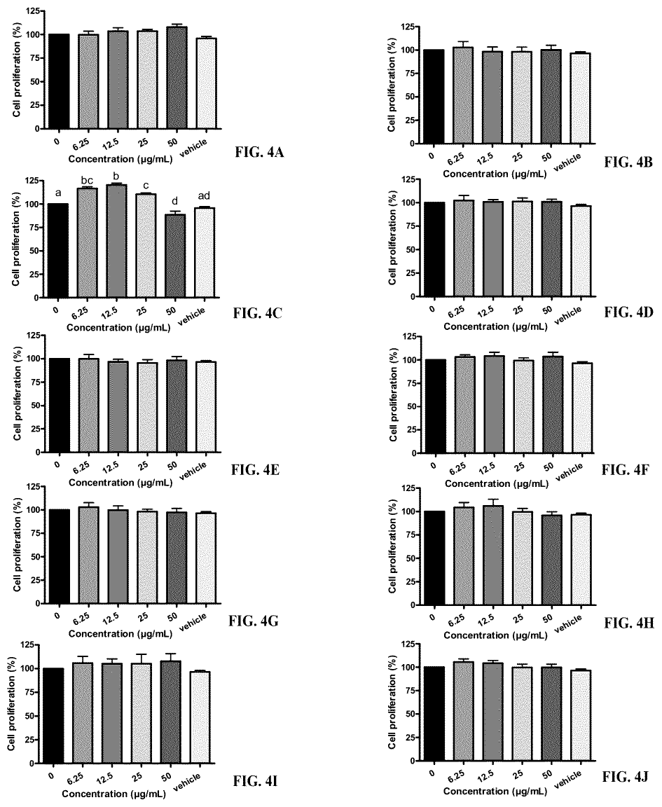

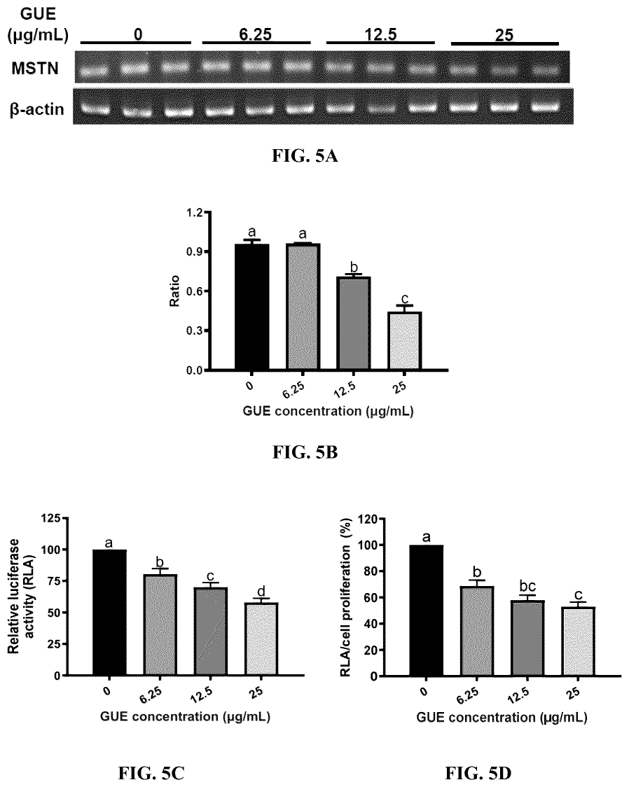

The results showed induced MSTN-Luc expression by dexamethasone at the effective concentrations reported previously (FIG. 3A). Thus, the MSTN-Luc activity in L8 MSTN-Luc cells can be used as a cell-based assay for MSTN expression in myoblast to detect the bioactivity of phytogenics, plant extracts, fractions or phytochemicals. Except for GUE (FIG. 4C), the other herbal extracts, i.e., A. paniculate (FIG. 4A), C. asiatica (FIG. 4B), G. pentaphyllum (FIG. 4D), P. grandifloras (FIG. 4E), P. chinense (FIG. 4F), P. oleracea (FIG. 4G), S. chinensis (FIG. 4H), S. china (FIG. 4I) and T. campylodes (FIG. 4J) did not increase L8 MSTN-Luc cell proliferation. The low-concentration GUE was found to have substantial and significant effects (p<0.05) on L8 MSTN-Luc cell proliferation, i.e., by about 15% to 20% increase (FIG. 4C). The similar patterns were observed from the luciferase activity results of L8 MSTN-Luc cells treated with various herbal extracts (FIG. 3). The MSTN mRNA level in L8 MSTN-Luc cells treated with GUE was analyzed by RT-PCR. The decreased mRNA level of MSTN in the GUE-treated groups was concentration-dependent. The higher concentration of GUE led to greater inhibition (p<0.05) of MSTN mRNA level in L8 MSTN-Luc cells (FIGS. 5A and 5B). Therefore, GUE with increased cell proliferation and luciferase inhibition (FIGS. 5C and 5D) was used for further in vitro studies.

### Example 2: Characterization and Identification of Active Fractions in GUE

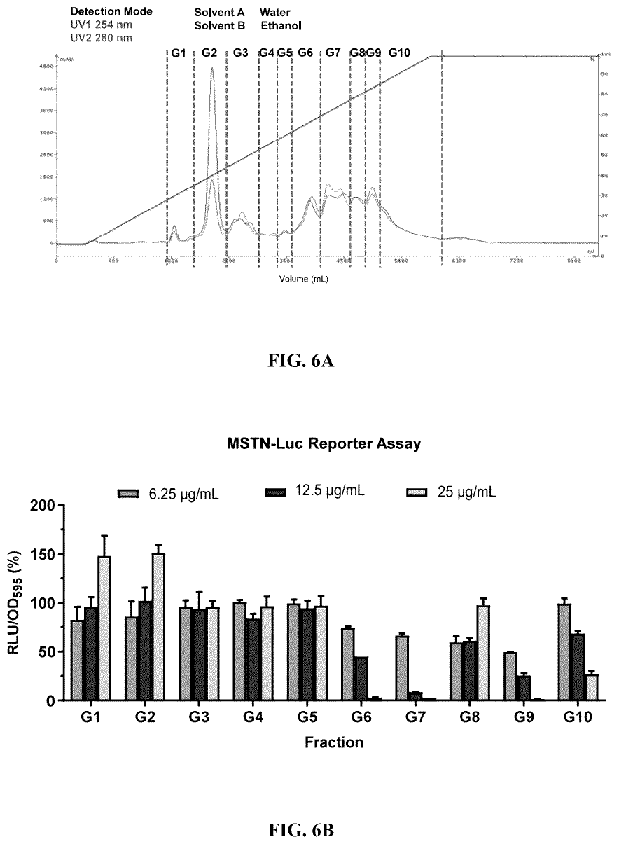

In this study, the process-scale liquid column chromatography was used to enrich biological activity of the GUE. The full-spectrum GUE was separated into 10 fractions (G1-G10) in a gradient elution mode (FIG. 6A), and the activity of each fraction was examined by MSTN-reporter assay in L8 MSTN-Luc cells (FIG. 6B). The G9 fraction was used for further purification and identification among the active fractions.

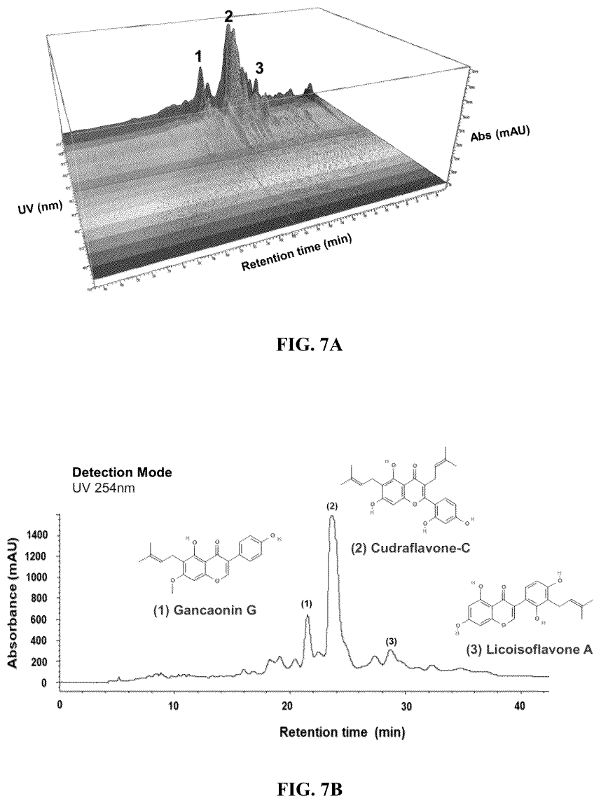

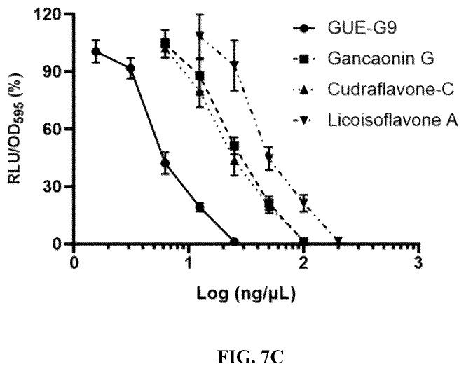

Next, the G9 fraction was further purified by column chromatography using an RP-18 column (FIG. 7A). Three bioactive phytocompounds were found in G9 fraction. Chemical structures of the bioactive compounds were then determined by nuclear magnetic resonance (NMR) and mass spectrometry (MS). As marked in the HPLC chromatogram, elution peaks that contained bioactive compounds and their respective chemical structures were identified as gancaonin G, cudraflavone-C, and licoisoflavone A (FIG. 7B). The efficacy and potency of each bioactive compound for MSTN suppression as determined by L8 MSTN-Luc cell-based reporter assay was concentration-dependent (FIG. 7C). The relative concentration of the maximal inhibition was GUE-G9<gancaonin G<cudraflavone-C<licoisoflavone A, and the inhibitory trends were almost the same in each treatment group.

### Example 3: Effect of GUE as a Feed Additive on Growth Performance in Growth-Finishing Pigs

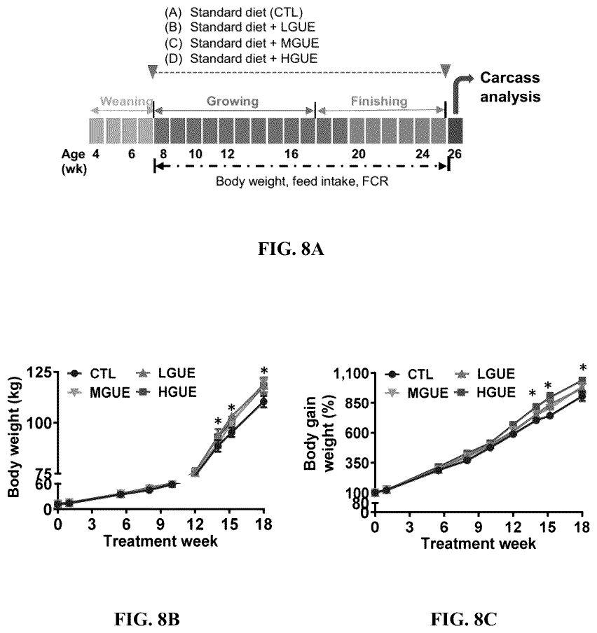

The effect of GUE as feed additives in growth-finishing pigs was investigated according to the timeline shown in the flow chart (FIG. 8A). After weaning at the age of 7 weeks, the pigs were randomized into four groups with 10 pigs per group, i.e., Group (A): standard control diet (“CTL”), Group (B): standard diet with the feed additive of low GUE concentration (“LGUE”), Group (C): standard diet with the feed additive of medium GUE concentration (“MGUE”), and Group (D): standard diet with the feed additive of high GUE concentration (“HGUE”). The pigs of Group (A) were fed with a standard diet for 18 weeks. The pigs of Group (B) were fed with a standard diet containing 0.01% GUE (LGUE) by weight of the diet for 18 weeks. The pigs of Group (C), were fed with a standard diet containing 0.05% GUE (MGUE) by weight of the diet for 18 weeks. The pigs of Group (D) were fed with a standard diet containing 0.1% GUE (HGUE) by weight of the diet for 18 weeks. The body weight and body gain weight in each treatment group was monitored before and after 18-week treatment.

According to the results after feeding, the final body weight (BW) showed a significant increase (p<0.05) in the GUE-treated group. In addition, the BW gains in the GUE-treated groups were significantly higher (p<0.05) than that in the control group (FIGS. 8B and 8C). There was a significant increase (p<0.05) in average daily gain (ADG) and decrease in feed conversion rate (FCR) in the GUE-treated groups, as shown in Table 1 below.

TABLE 1
Effect of GUE as a feed additive on the parameter of growth performance in pigs

| Treatment group | CTL | LGUE | MGUE | HGUE |
|---|---|---|---|---|
| Initial body weight (kg) | 11.73 ± 1.22 | 12.62 ± 1.62 | 12.89 ± 1.18 | 11.40 ± 1.18 |
| Final body weight (kg) | 110.38 ± 6.29 ^{a} | 118.68 ± 8.12 ^{b} | 119.48 ± 6.54 ^{b} | 118.17 ± 10.91 ^{b} |
| Body weight gain (kg) | 98.67 ± 7.38 ^{a} | 106.06 ± 7.48 ^{b} | 106.59 ± 6.43 ^{b} | 106.77 ± 9.83 ^{b} |
| ADG (kg/day) | 0.78 ± 0.06 ^{a} | 0.84 ± 0.06 ^{b} | 0.85 ± 0.05 ^{b} | 0.85 ± 0.08 ^{b} |
| ADFI (kg/day) | 2.00 ± 0.57 | 2.00 ± 0.42 | 2.00 ± 0.75 | 1.98 ± 0.82 |
| TFI (kg) | 257.31 ± 7.91 | 256.92 ± 8.32 | 257.17 ± 6.11 | 255.16 ± 5.43 |
| FCR | 2.61 ^{a} | 2.42 ^{b} | 2.41 ^{b} | 2.39 ^{b} |

Four groups of weaned pigs were fed standard control diet (CTL), or standard diet containing low GUE concentration (LGUE), medium GUE concentration (MGUE), or high GUE concentration (HGUE) for 18 weeks. ADG: average daily gain, ADFI: average daily feed intake, TFI: total feed intake, and FCR: feed conversion rate. Each data point represents the mean ± SEM. Different letters (^{a}, ^{b}) denotes statistical significance (P < 0.05) among groups.

The effect of GUE as a feed additive on carcass characteristics of pigs was also investigated according to the timeline (FIG. 8A). After weaning at the age of 7 weeks, the pigs were randomized into four groups with 6 pigs per group, i.e., Group (A): standard control diet (“CTL”), Group (B): standard diet with the feed additive of low GUE concentration (“LGUE”), Group (C): standard diet with the feed additive of medium GUE concentration (“MGUE”), and Group (D): standard diet with the feed additive of high GUE concentration (“HGUE”). The pigs of Group (A) were fed with a standard diet for 18 weeks. The pigs of Group (B) were fed with a standard diet containing 0.01% GUE (LGUE) by weight of the diet for 18 weeks. The pigs of Group (C) were fed with a standard diet containing 0.05% GUE (MGUE) by weight of the diet for 18 weeks. The pigs of Group (D) were fed with a standard diet containing 0.1% GUE (HGUE) by weight of the diet for 18 weeks. The carcass weight, carcass percentage, carcass length, and the percentage of lean, fat and skin in each treatment group was measured and calculated at the end of 18 weeks.

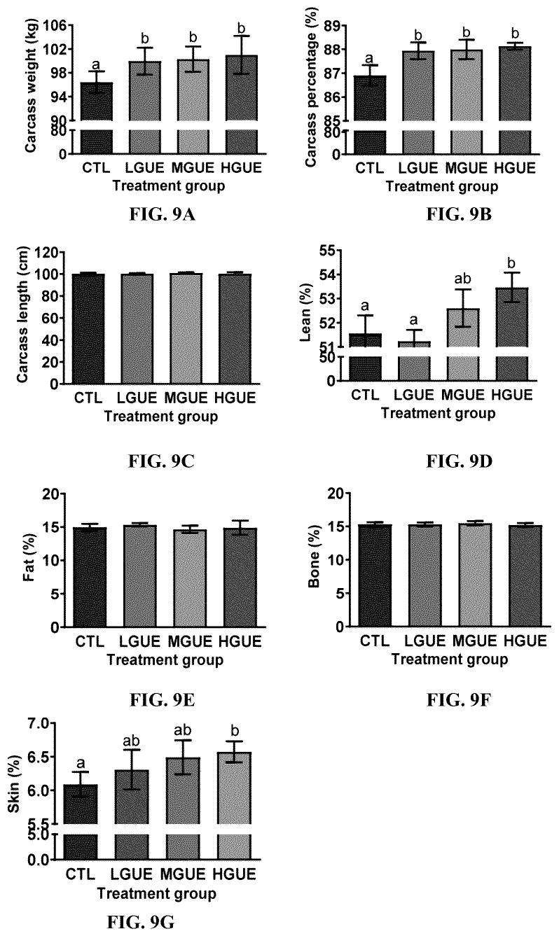

According to the results, the carcass weight and carcass percentage in the GUE-treated groups were significantly higher (p<0.05) than those in the control group (FIGS. 9A and 9B), while there was no significant difference (p>0.05) in carcass length among groups (FIG. 9C). The lean and skin percentages of carcass were significant higher (p<0.05) in the GUE-treated groups than the control group (FIGS. 9D and 9G), while there was no significant difference (p>0.05) in fat and bone percentages of carcass among groups (FIGS. 9E and 9F).

### Example 4: Effect of GUE as a Feed Additive on Gut Microbiota in Growth-Finishing Pigs

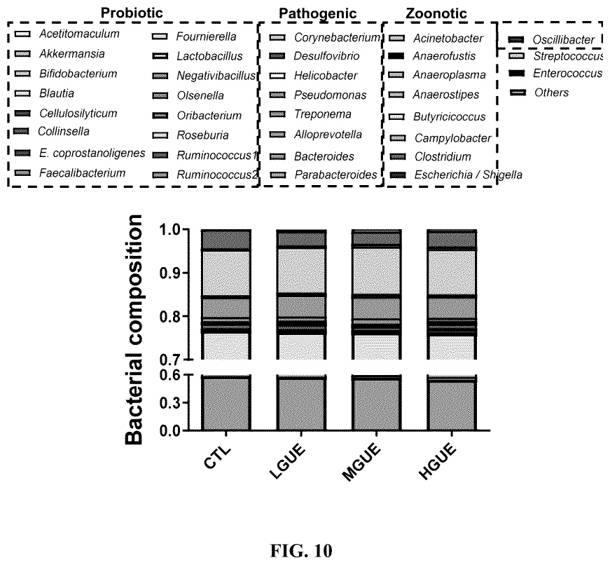

The correlation between gut microbiota and growth performance in pigs have been reported (Recharla, N. et al., J. Anim. Sci. Technol. 2022, 64, 640-653). Therefore, the correlation between microbiota and growth performance in pigs fed with GUE was investigated. The pigs in each of the four groups were fed with control diet (CTL), or diet containing low (0.01%) GUE concentration (LGUE), medium (0.05%) GUE concentration (MGUE) and high (0.1%) GUE concentration (HGUE) for 18 weeks. The bacterial composition at the genus level in the guts of the three representative pigs were pyrosequenced and the data were analyzed at the end of 18 weeks. The results from bacterial composition in pig gut (FIG. 10) showed the pathogenic and zoonotic bacteria genera did not differ significantly (p>0.05) in the guts of pigs fed standard diet with or without GUE. Thus, GUE may not increase growth performance through gut microbiota regulation.

### Conclusion

The results of the above studies indicated that the plant-based or phytogenic feed additives enhance animal health and growth performance. Specifically, the results in this study showed that G. uralensis extract was the only tested plant extract capable of increasing cell proliferation (FIG. 4C) and inhibiting MSTN expression in L8 cells (FIG. 5A). It was also observed that the luciferase activity decrease (FIG. 5B), and the ratio of luciferase activity to cell proliferation was used to evaluate effect of GUE on MSTN expression in L8 cells (FIG. 5C). MSTN has been known to inhibit myogenesis through inhibition of myoblast proliferation, as reported in Molfino, A. et al., Expert Opinion on Biological Therapy 2016, 16, 1239-1244; Lee, S.-J. The Journal of clinical investigation 2021, 131. According to the present disclosure, G. uralensis extract suppressed MSTN expression and directly promoted L8 myoblast proliferation. These results suggested that G. uralensis extract has the potential as a feed additive and contain active comportments to promote myogenesis. The process-scale liquid column chromatography was then used to enrich the biological activity of the G. uralensis extract and identify possible active components thereof. Using MSTN activity in L8 MSTN-Luc cells as the evaluation method, the efficacy and potency for suppressing MSTN activity in all three phytocompounds were investigated (FIGS. 6 and 7). The results showed that these three active compounds, i.e., gancaonin G, cudraflavone-C, and licoisoflavone A, suppressed MSTN activity and increased cell proliferation. However, the G. uralensis extract of G9 fraction was more effective than these three active compounds (FIG. 7C) due to the combination effect of these compounds.

Furthermore, to investigate the potential of G. uralensis extract as a feed additive, the growth-finishing pigs were fed diets containing different GUE concentration. The body weight and weight gain percentage were significantly increased in the G. uralensis extract-treated groups (FIGS. 8B and 8C). As G. uralensis extract showed myogenesis effect in growth-finishing pigs (FIG. 8), the increased body weight gain in pigs fed with G. uralensis extract may partially be due to the increase of lean percentage (FIG. 9D). This result showed that the concentration-dependent increase in lean percentage was observed in the G. uralensis extract-treated groups. Therefore, higher concentration of G. uralensis extract led to greater improvements in lean percentage. There was no significant different in average daily feed intake (ADFI) and total feed intake (TFI) with or without treatment with G. uralensis extract as shown in Table 1. However, significant differences were present in average daily gain (ADG) and feed conversion rate (FCR) between the control and G. uralensis extract-treated groups. By modulating MSTN expression in skeletal muscles of an animal, G. uralensis extract can prompt animal growth by increasing body weight and lean content of the animal. Consequently, due to beneficial effects of G. uralensis extract, this observation suggests that G. uralensis extract potentially serve as a favorable feed additive for boosting animal growth under current animal production system.

Akin to most clinical medicine, medical herbs, phytogenic and phytocompounds have been used in animals through multiple mechanisms, including regulation of cell development, metabolism of nutrients and bacterial composition of gut microbiota, etc. Other researches also reported that G. uralensis-related products as feed additives inhibit the relative amount of Enterobacter, Enterococcus, Clostridium and Bacteroides and increased the relative abundance of Lactobacillus at genus levels in weaned piglets. In the present disclosure, gut microbiota was analyzed to evaluate the relative amount of probiotic as well as pathogenic and zoonotic bacteria genera in growth-finishing pigs, indicated that GUE did not alter the bacterial composition gut microbiota in growth-finishing pigs (FIG. 10). Pigs at the growth-finishing stage display heightened maturity in contrast to their counterparts at the weaned stage. Consequently, the gut microbiota of growth-finishing pigs may exhibit greater stability than that of weaned piglets. On the other hand, due to the brief 4 to 5-week span during the weaned piglet stage, the impact of feed additives on gut microbiota during this stage could be relatively effective. In this study, the animals fed with G. uralensis extract as a feed additive for 18 weeks did not show significant influence on the gut microbiota of growth-finishing pigs. Accordingly, the results revealed that the observed G. uralensis extract-enhanced growth performance may not be due to gut microbiota regulation.

These data suggest a novel application for G. uralensis as a feed additive in growth performance enhancement through modulation of MSTN expression. In summary, the edible plant, G. uralensis, is recognized as safe for ethnomedicinal or culinary use worldwide, and the findings from this study along with the published literature confirm the beneficial function and effects of G. uralensis extract on animal growth.

The results from the established systematic rodent myoblast screening platform demonstrated the effectiveness in identifying phytogenic compounds by evaluating myostatin promoter activity. Through this screening platform, the G. uralensis extract was identified and found to significantly increase body weight, carcass weight, and lean content in pigs. In conclusion, from the rodent myoblast screening platform, the G. uralensis extract successfully identified as a myogenesis-promoting phytogenic and the active fractions from the G. uralensis extract inhibited MSTN expression. These findings suggest a novel role of G. uralensis extract in enhancing growth performance in economic animals by modulating MSTN expression. Furthermore, the screening platform or method established in this study also exhibit a great potential for evaluating a wide variety of phytogenics related to myogenesis.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All references cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

### REFERENCES

ADDIN EN.REFLIST 1. Giordani, L.; He, G. J.; Negroni, E.; Sakai, H.; Law, J. Y. C.; Siu, M. M.; Wan, R.; Corneau, A.; Tajbakhsh, S.; Cheung, T. H., et al. High-Dimensional Single-Cell Cartography Reveals Novel Skeletal Muscle-Resident Cell Populations. Molecular Cell 2019, 74, 609-621.
2. Tieland, M.; Trouwborst, I.; Clark, B. C. Skeletal muscle performance and ageing. Journal of Cachexia, Sarcopenia and Muscle 2018, 9, 3-19.
3. Matarneh, S. K.; Silva, S. L.; Gerrard, D. E. New Insights in Muscle Biology that Alter Meat Quality. Annual Review of Animal Biosciences 2021, 9, 355-377.
4. Niranjan, D.; Sridhar, N.; Chandra, U.; Manjunatha, S.; Borthakur, A.; Vinuta, M.; Mohan, B. Recent perspectives of growth promoters in livestock: an overview. Journal of Livestock Science (ISSN online 2277-6214) 2023, 14, 53-64.
5. Elliott, C. T.; Crooks, S. R.; McEvoy, J. G.; McCaughey, W. J.; Hewitt, S. A.; Patterson, D.; Kilpatrick, D. Observations on the effects of long-term withdrawal on carcass composition and residue concentrations in clenbuterol-medicated cattle. Vet Res Commun 1993, 17, 459-46.
6. Moseley, W. M.; Paulissen, J. B.; Goodwin, M. C.; Alaniz, G. R.; Claflin, W. H. Recombinant bovine somatotropin improves growth performance in finishing beef steers. J Anim Sci 1992, 70, 412-425.
7. Martinez-Navarro, J. F. Food poisoning related to consumption of illicit β-agonist in liver. The Lancet 1990, 336, 1311.
8. Lehmann, S.; Thomas, A.; Schiwy-Bochat, K.-H.; Geyer, H.; Thevis, M.; Glenewinkel, F.; Rothschild, M. A.; Andresen-Streichert, H.; Juebner, M. Death after misuse of anabolic substances (clenbuterol, stanozolol and metandienone). Forensic Science International 2019, 303, 109925.
9. Skoupá, K.; Št'astný, K.; Sládek, Z. Anabolic Steroids in Fattening Food-Producing Animals&mdash; A Review. Animals 2022, 12, 2115.
10. Hirpessa, B. B.; Ulusoy, B. H.; Hecer, C. Hormones and hormonal anabolics: residues in animal source food, potential public health impacts, and methods of analysis. Journal of Food Quality 2020, 2020, 1-12.
11. Martel, J.; Ojcius, D. M.; Ko, Y-F.; Young, J. D. Phytochemicals as Prebiotics and Biological Stress Inducers. Trends in Biochemical Sciences 2020, 45, 462-471.
12. Dhama, K.; Karthik, K.; Khandia, R.; Munjal, A.; Tiwari, R.; Rana, R.; Khurana, S. K.; Sana, U.; Khan, R. U.; Alagawany, M., et al. Medicinal and Therapeutic Potential of Herbs and Plant Metabolites/Extracts Countering Viral Pathogens—Current Knowledge and Future Prospects. Curr Drug Metab 2018, 19, 236-263.
13. Abdelli, N.; Solà-Oriol, D.; Pérez, J. F. Phytogenic Feed Additives in Poultry: Achievements, Prospective and Challenges. Animals 2021, 11, 3471.
14. Upadhaya, S. D.; Kim, I. H. Efficacy of Phytogenic Feed Additive on Performance, Production and Health Status of Monogastric Animals—A Review. Annals of Animal Science 2017, 17, 929-948.
15. Windisch, W.; Schedle, K.; Plitzner, C.; Kroismayr, A. Use of phytogenic products as feed additives for swine and poultry1. Journal of Animal Science 2008, 86, E140-E148.
16. Mnisi, C. M.; Mlambo, V.; Gila, A.; Matabane, A. N.; Mthiyane, D. M. N.; Kumanda, C.; Manyeula, F.; Gajana, C. S. Antioxidant and Antimicrobial Properties of Selected Phytogenics for Sustainable Poultry Production. Applied Sciences 2023, 13, 99.
17. Basiouni, S.; Tellez-Isaias, G.; Latorre, J. D.; Graham, B. D.; Petrone-Garcia, V. M.; El-Seedi, H. R.; Yalçin, S.; El-Wahab, A. A.; Visscher, C.; May-Simera, H. L., et al. Anti-Inflammatory and Antioxidative Phytogenic Substances against Secret Killers in Poultry: Current Status and Prospects. Veterinary Sciences 2023, 10, 55.
18. Marks, H.; Grześkowiak, Ł.; Martinez-Vallespin, B.; Dietz, H.; Zentek, J. Porcine and Chicken Intestinal Epithelial Cell Models for Screening Phytogenic Feed Additives&mdash; Chances and Limitations in Use as Alternatives to Feeding Trials. Microorganisms 2022, 10, 629.
19. McPherron, A. Regulation of skeletal muscle mass in mice by a new TGF-beta superfamily member.—p. 83-90; En: Nature (London)(United Kingdom).—Vol. 387, no. 6628 (1997): 1997.
20. Lee, S.-J. REGULATION OF MUSCLE MASS BY MYOSTATIN. Annual Review of Cell and Developmental Biology 2004, 20, 61-86.
21. Kambadur, R.; Sharma, M.; Smith, T. P.; Bass, J. J. Mutations in myostatin (GDF8) in double-muscled Belgian Blue and Piedmontese cattle. Genome research 1997, 7, 910-915.
22. Grisolia, A.; D'Angelo, G.; Porto Neto, L.; Siqueira, F.; Garcia, J. F. Myostatin (GDF8) single nucleotide polymorphisms in Nellore cattle. Genetics and molecular research 2009, 822-830.
23. Dilger, A. C.; Gabriel, S.; Kutzler, L.; McKeith, F.; Killefer, J. The myostatin null mutation and clenbuterol administration elicit additive effects in mice. animal 2010, 4, 466-471.
24. Salerno, M. S.; Thomas, M.; Forbes, D.; Watson, T.; Kambadur, R.; Sharma, M. Molecular analysis of fiber type-specific expression of murine myostatin promoter. American journal of physiology-Cell physiology 2004, 287, C1031-C1040.
25. Yeh, J. Y; Cheng, L. C.; Ou, B. R.; Whanger, D. P.; Chang, L. W. Differential influences of various arsenic compounds on glutathione redox status and antioxidative enzymes in porcine endothelial cells. Cell Mol Life Sci 2002, 59, 1972-1982.
26. Tahara, S.; Orihara, S.; Ingham, J. L.; Mizutani, J. Seventeen isoglavonoids from Lupinus albus roots. Phytochemistry 1989, 28, 901-911.
27. He, J.; Chen L.; Heber, D.; Shi, W.; Lu, Q.-Y. Antibacterial Compounds from Glycyrrhiza uralensis. J. Nat. Prod. 2006, 69, 121-124.
28. Dej-adisai, S.; Meechai, I; Puripattanavong, J.; Kummee, S. Antityroinase and antimicrobial activities from Thai medicinal plants. Arch. Pharm. Res. 2014, 37, 473-483.
29. Tung, H. Y; Chen, WC.; Ou, B. R.; Yeh, J. Y; Cheng, Y. H.; Tsng, P. H.; Hsu, M. H.; Tsai, M. S.; Liang, Y. C. Simultaneous detection of multiple pathogens by multiplex PCR coupled with DNA biochip hybridization. Lab Anim 2018, 52, 186-195.
30. Branscheid, W.; Dobrowolski, A.; Sack, E. Simplification of the EC reference method for the full dissection of pig carcasses. Fleischwirtschaft 1990, 70, 565-567.
31. Salehian, B.; Mahabadi, V.; Bilas, J.; Taylor, W. E.; Ma, K. The effect of glutamine on prevention of glucocorticoid-induced skeletal muscle atrophy is associated with myostatin suppression. Metabolism 2006, 55, 1239-1247.
32. Qin, J.; Du, R.; Yang, Y-Q.; Zhang, H.-Q.; Li, Q.; Liu, L.; Guan, H.; Hou, J.; An, X.-R. Dexamethasone-induced skeletal muscle atrophy was associated with upregulation of myostatin promoter activity. Research in veterinary science 2013, 94, 84-89.
33. Sankar, V.; SalinRaj, P.; Athira, R.; Soumya, R. S.; Raghu, K. G. Cerium nanoparticles synthesized using aqueous extract of Centella asiatica: characterization, determination of free radical scavenging activity and evaluation of efficacy against cardiomyoblast hypertrophy. RSC advances 2015, 5, 21074-21083.
34. Yoshioka, Y; Yamashita, Y; Kishida, H.; Nakagawa, K.; Ashida, H. Licorice flavonoid oil enhances muscle mass in KK-Ay mice. Life Sciences 2018, 205, 91-96.
35. Chi, A.; Tang, L.; Zhang, J.; Zhang, K. Chemical composition of three polysaccharides from Gynostemma pentaphyllum and their antioxidant activity in skeletal muscle of exercised mice. International Journal of Sport Nutrition and Exercise Metabolism 2012, 22, 479-485.
36. Yoon, H. J.; Bang, M.-H.; Kim, H.; Imm, J.-Y Improvement of palmitate-induced insulin resistance in C2C12 skeletal muscle cells using Platycodon grandiflorum seed extracts. Food Bioscience 2018, 25, 61-67.
37. Zhou, M.; Li, J.; Wu, J.; Yang, Y; Zeng, X.; Lv, X.; Cui, L.; Yao, W.; Liu, Y Preventive effects of Polygonum multiflorum on glucocorticoid-induced osteoporosis in rats. Experimental and Therapeutic Medicine 2017, 14, 2445-2460.
38. Parry, O.; Okwuasaba, F.; Ejike, C. Skeletal muscle relaxant action of an aqueous extract of Portulaca oleracea in the rat. Journal of ethnopharmacology 1987, 19, 247-253.
39. Jung, M.-H.; Song, M.-C.; Bae, K.; Kim, H. S.; Kim, S. H.; Sung, S. H.; Ye, S. K.; Lee, K. H.; Yun, Y-P.; Kim, T.-J. Sauchinone attenuates oxidative stress-induced skeletal muscle myoblast damage through the down-regulation of ceramide. Biological and Pharmaceutical Bulletin 2011, 34, 575-579.
40. Amaro, C. A. B.; González-Cortazar, M.; Herrera-Ruiz, M.; Román-Ramos, R.; Aguilar-Santamaria, L.; Tortoriello, J.; Jiménez-Ferrer, E. Hypoglycemic and Hypotensive Activity of a Root Extract of Smilax aristolochiifolia, Standardized on N-trans-Feruloyl-Tyramine. Molecules 2014, 19, 11366-11384.
41. Liu, Q.; Zhao, H.; Gao, Y; Meng, Y; Zhao, X.-X.; Pan, S.-N. Effects of dandelion extract on the proliferation of rat skeletal muscle cells and the inhibition of a lipopolysaccharide-induced inflammatory reaction. Chinese Medical Journal 2018, 131, 1724-1731.
42. Recharla, N.; Park, S.; Kim, M.; Kim, B.; Jeong, J. Y Protective effects of biological feed additives on gut microbiota and the health of pigs exposed to deoxynivalenol: a review. J Anim Sci Technol 2022, 64, 640-653.
43. Shehata, A. A.; Yalgin, S.; Latorre, J. D.; Basiouni, S.; Attia, YA.; Abd El-Wahab, A.; Visscher, C.; El-Seedi, H. R.; Huber, C.; Hafez, H. M., et al. Probiotics, Prebiotics, and Phytogenic Substances for Optimizing Gut Health in Poultry. Microorganisms 2022, 10, 395.
44. Suh, J.; Lee, Y-S. Myostatin inhibitors: panacea or predicament for musculoskeletal disorders? Journal of bone metabolism 2020, 27, 151.
45. Molfino, A.; Amabile, M. I.; Rossi Fanelli, F.; Muscaritoli, M. Novel therapeutic options for cachexia and sarcopenia. Expert Opinion on Biological Therapy 2016, 16, 1239-1244.
46. Lee, S.-J. Targeting the myostatin signaling pathway to treat muscle loss and metabolic dysfunction. The Journal of clinical investigation 2021, 131.
47. Ryan, D. H. Next Generation Antiobesity Medications: Setmelanotide, Semaglutide, Tirzepatide and Bimagrumab: What do They Mean for Clinical Practice? J Obes Metab Syndr 2021, 30, 196-208, doi:10.7570/jomes21033.
48. Joulia, D.; Bernardi, H.; Garandel, V.; Rabenoelina, F.; Vernus, B.; Cabello, G. Mechanisms involved in the inhibition of myoblast proliferation and differentiation by myostatin. Experimental Cell Research 2003, 286, 263-275.
49. Thomas, M.; Langley, B.; Berry, C.; Sharma, M.; Kirk, S.; Bass, J.; Kambadur, R. Myostatin, a Negative Regulator of Muscle Growth, Functions by Inhibiting Myoblast Proliferation. Journal of Biological Chemistry 2000, 275, 40235-40243
50. Wang, Y; Shou, J.-W.; Li, X.-Y; Zhao, Z.-X.; Fu, J.; He, C.-Y; Feng, R.; Ma, C.; Wen, B.-Y; Guo, F. Berberine-induced bioactive metabolites of the gut microbiota improve energy metabolism. Metabolism 2017, 70, 72-84.
51. Jia, Q.; Wang, L.; Zhang, X.; Ding, Y; Li, H.; Yang, Y; Zhang, A.; Li, Y; Lv, S.; Zhang, J. Prevention and treatment of chronic heart failure through traditional Chinese medicine: Role of the gut microbiota. Pharmacological Research 2020, 151, 104552.
52. Zhang, C.; Li, C.; Zhao, P.; Shao, Q.; Ma, Y; Bai, D.; Liao, C.; He, L.; Huang, S.; Wang, X. Effects of dietary Glycyrrhiza polysaccharide supplementation on growth performance, intestinal antioxidants, immunity and microbiota in weaned piglets. Animal Biotechnology 2022, 10.1080/10495398.2022.2086878, 1-12.
53. Kim, H.-Y; Zuo, G.; Lee, S. K.; Lim, S. S. Acute and subchronic toxicity study of nonpolar extract of licorice roots in mice. Food Science & Nutrition 2020, 8, 2242-2250.
54. Wang, X.; Zhang, H.; Chen, L.; Shan, L.; Fan, G.; Gao, X. Liquorice, a unique “guide drug” of traditional Chinese medicine: A review of its role in drug interactions. Journal of Ethnopharmacology 2013, 150, 781-790.

We claim:

1. A composition comprising Glycyrrhiza uralensis extract or an active compound isolated from the G. uralensis extract.

2. The composition according to claim 1, wherein the G. uralensis extract or the active compound isolated from the G. uralensis extract is present in an amount effective to promote skeletal muscle growth in an animal in need thereof.

3. The composition according to claim 1, wherein the G. uralensis extract is present at a concentration ranging from about 5 μg/mL to about 30 μg/mL.

4. The composition according to claim 1, wherein the G. uralensis extract is present at a concentration ranging from about 6.25 μg/mL to about 25 μg/mL.

5. The composition according to claim 1, wherein the G. uralensis extract is present at a concentration ranging from about 12.5 μg/mL to about 25 μg/mL.

6. The composition according to claim 1, wherein the G. uralensis extract is present at a concentration of about 12.5 μg/mL or about 25 μg/mL.

7. The composition according to claim 1, wherein the active compound isolated from G. uralensis extract in the composition of the present disclosure is selected from the group consisting of gancaonin G, cudraflavone-C and licoisoflavone A.

8. A method for promoting skeletal muscle growth in an animal in need thereof, comprising administering to the animal a composition in an animal feed, wherein the composition comprises G. uralensis extract or an active compound isolated from the G. uralensis extract.

9. The method according to claim 8, wherein the G. uralensis extract is present at a concentration ranging from about 5 μg/mL to about 30 μg/mL.

10. The method according to claim 8, wherein the G. uralensis extract is present at a concentration ranging from about 6.25 μg/mL to about 25 μg/mL.

11. The method according to claim 8, wherein the G. uralensis extract is present at a concentration ranging from about 12.5 μg/mL to about 25 μg/mL.

12. The method according to claim 8, wherein the G. uralensis extract is present at a concentration of about 6.25 μg/mL, about 12.5 μg/mL, or about 25 μg/mL.

13. The method according to claim 8, wherein the active compound isolated from G. uralensis extract is selected from the group consisting of gancaonin G, cudraflavone-C and licoisoflavone A.

14. The method according to claim 8, wherein the composition when comprises G. uralensis extract in an amount ranging from about 0.01% to about 0.1% by weight of the feed.

15. The method according to claim 8, wherein the composition when comprises G. uralensis extract in an amount ranging from about 0.01% to about 0.05% by weight of the feed.

16. The method according to claim 8, wherein the composition when comprises G. uralensis extract in an amount ranging from about 0.05% to about 0.1% by weight of the feed.

17. The method according to claim 8, wherein the composition when comprises G. uralensis extract in an amount of about 0.01%, about 0.5%, or 0.1% by weight of the feed.

18. Use of a composition comprising G. extract or an active compound isolated from the G. uralensis extract as a feed additive for promoting skeletal muscle growth in an animal in need thereof.

19. The use according to claim 18, wherein the G. uralensis extract is present at a concentration ranging from about 5 μg/mL to about 30 μg/mL.

20. The use according to claim 18, wherein the G. uralensis extract is present at a concentration ranging from about 6.25 μg/mL to about 25 μg/mL.

21. The use according to claim 18, wherein the G. uralensis extract is present at a concentration ranging from about 12.5 μg/mL to about 25 μg/mL.

22. The use according to claim 18, wherein the G. uralensis extract is present at a concentration of about 6.25 μg/mL, about 12.5 μg/mL, or about 25 μg/mL.

23. The use according to claim 18, wherein the active compound isolated from G. uralensis extract is selected from the group consisting of gancaonin G, cudraflavone-C and licoisoflavone A.

24. The use according to claim 18, wherein the composition when comprises G. uralensis extract in an amount ranging from about 0.01% to about 0.1% by weight of the feed.

25. The use according to claim 18, wherein the composition when comprises G. uralensis extract in an amount ranging from about 0.01% to about 0.05% by weight of the feed.

26. The use according to claim 18, wherein the composition when comprises G. uralensis extract in an amount ranging from about 0.05% to about 0.1% by weight of the feed.

27. The use according to claim 18, wherein the composition when comprises G. uralensis extract in an amount of about 0.01%, about 0.5%, or 0.1% by weight of the feed.

28. An animal feed additive comprising a composition according to claim 1.

29. A screening method for a potential phytogenic in promoting skeletal muscle growth in animals comprises:
(a) preparing a plant extract from a plant or plant part of interest;
(b) preparing the animal cells by transfecting the cells with a plasmid containing a myostatin-luciferase (MSTN-Luc) reporter gene;
(c) treating the transfected animal cells from (b) with the herbal extract from (a) and maintaining the treated cells in a culture medium under a condition sufficient for cell proliferation;
(d) measuring cell proliferation of the treated cells from (c);
(e) measuring the luciferase activity of the treated cells from (c);
(f) comparing the cell proliferation measured in (d) with a vehicle control under the same condition;
(g) comparing the luciferase activity measured in (e) with a control under the same condition; and
(h) determining the plant extract tested as effective phytogenic for promoting skeletal muscle growth by the increased cell proliferation and higher inhibition of luciferase activity exhibited by the cells treated with the plant extract.

30. The screening method according to claim 29, wherein the animal cells prepared in (b) are rat L8 myoblast cell line.

31. The screening method according to claim 29, wherein the cell proliferation of the treated cells in (d) is measured by performing MTT (3-(4,5-dimethylthiazolyl-2)-2,5-diphenyltetrazolium bromide) colorimetric assay.

32. The screening method according to claim 31, wherein MTT reagent is added to the treated cells cultivated in a culture medium at 37° C. for 4 h, followed by adding dimethyl sulfoxide (DMSO) to detect optical density at 595 nm.

33. The screening method according to claim 29, wherein the luciferase activity of the treated cells incubated in a culture medium is measured in (e) after 24 h treatment.


SEQUENCE LISTING (6 sequences)

SEQ ID NO: 1 - DNA, 34 bp, synthetic construct
  source 1..34: mol_type = other DNA
ataagatctcctttttaagtcctaagtcacacgg

SEQ ID NO: 2 - DNA, 29 bp, synthetic construct
  source 1..29: mol_type = other DNA
ataggatccccagggagtcctgtatactg

SEQ ID NO: 3 - DNA, 20 bp, synthetic construct
  source 1..20: mol_type = other DNA
ccaggcactggtatttggca

SEQ ID NO: 4 - DNA, 20 bp, synthetic construct
  source 1..20: mol_type = other DNA
aagtctctccgggacctctt

SEQ ID NO: 5 - DNA, 21 bp, synthetic construct
  source 1..21: mol_type = other DNA
ggttggtatgggccagaaaga

SEQ ID NO: 6 - DNA, 23 bp, synthetic construct
  source 1..23: mol_type = other DNA
tggtgacaataccgtgttcaatg